(19) United States
(12) Patent Application Publication
(10) Pub. No.: US 2026/0306851 A1
(43) Pub. Date: Oct. 8, 2026

(54) HIGH-RESOLUTION MICROELECTRODE-BASED NEURAL STIMULATION SYSTEM

(71) Applicant: ACADEMIA SINICA, Taipei City (TW)

(72) Inventors: Yu-Wei Wu, Taipei City (TW); Hao-Tung Yang, Taipei City (TW); Yu-Tsao Hsing, Taipei City (TW)

(21) Appl. No.: 19/483,465

(22) PCT Filed: May 9, 2024
(86) PCT No.: PCT/US2024/028581

§ 371 Date: Nov. 11, 2025

Related U.S. Application Data
(60) Provisional application No. 63/465,947, filed on May 12, 2023.

(51) Int. Cl. A61N 1/36; A61N 1/05

(52) U.S. Cl. CPC A61N 1/36171; A61N 1/0529; A61N 1/3615; A61N 1/36185

(57) ABSTRACT

A high-resolution microelectrode-based neural stimulation system includes at least two stimulating electrodes, a first control module, and a second control module. An overall periphery of the stimulating electrodes defines a stimulation area on the brain tissue. The first control module has a first signal source and at least two first switches. The first signal source provides a first current having a first frequency. The second control module has a second signal source and at least two second switches. The second signal source provides a second current having a second frequency. By controlling the current intensities of any of the two electrodes, stimulation can be selectively provided to a brain tissue on a target site in the stimulation area. The neural stimulation system benefits its user with decreased complexity of electrode installation and reduced setup costs while enabling high-precision stimulation to neurons.

### BACKGROUND OF THE INVENTION

### 1. Field of the Invention

The present invention relates to neural stimulation and, more particularly, to a high-spatiotemporal resolution microelectrode-based neural stimulation system.

### 2. Description of the Related Art

Neurons play a crucial role in various brain functions, such as decision-making, motor control, and memory consolidation. Recent research has shown that neural stimulation has great potential in treating neurological conditions such as Parkinson's disease and in applications related to brain-machine interfaces for prosthesis control.

Stimulation with single-cell resolution can be achieved through microelectrode-based or two-photon optogenetic approaches. Microelectrode-based stimulation requires moving electrodes among neurons. However, since the brain operates on a millisecond-level resolution, it is obviously impracticable to move electrodes one by one to stimulate the corresponding neurons. A high-density microelectrode array can deliver neural stimulation at multiple sites without moving electrodes in the tissue. However, the spatial resolution of the stimulation is limited by the density of the electrode, i.e., the distance between the electrodes.

### SUMMARY OF THE INVENTION

The primary objective of the present invention is to address the shortcomings of microelectrode-based stimulation in the prior art about being inferior in terms of spatial resolution and requiring a full-coverage microelectrode array.

To achieve the foregoing objective, one embodiment of the present invention provides a high-resolution microelectrode-based neural stimulation system, for providing electrical stimulation to a brain tissue, the microelectrode-based neural stimulation system comprising: at least two stimulating electrodes, being fixed to and contacting the brain tissue so that between two stimulating electrodes defines a stimulation area on the brain tissue; a first control module, having a first signal source and at least two first switches, the first signal source providing a first current having a first frequency, each of the first switches corresponding to a respective one of the stimulating electrodes and being electrically connected to the first signal source, so as to respectively control each of the stimulating electrodes to form electric conduction with the first signal source; and a second control module, having a second signal source and at least two second switches, the second signal source providing a second current having a second frequency that is different from the first frequency, each of the second switches corresponding to a respective one of the stimulating electrodes and being electrically connected to the second signal source, so as to respectively control each of the stimulating electrodes to form electric conduction with the second signal source; whereby the first control module controls one of the first switches to form electric conduction between the corresponding stimulating electrode and the first signal source, so as to transmit the first current to the brain tissue, and the second control module controls one of the second switches to form electric conduction between another of the stimulating electrodes and the second signal source, so as to transmit the second current to the brain tissue, in which the first control module and the second control module jointly form an electrical stimulation signal on a target site in the stimulation area by controlling current intensities of the first current and the second current so as to provide the electrical stimulation to the brain tissue.

Thereby, with the designed arrangement of those stimulating electrodes, the disclosed microelectrode-based neural stimulation system can use the first control module and the second control module to respectively control any two of the stimulating electrodes so as to form an electrical stimulation signal on a target site in a stimulation area and provide stimulation to a neuron on the target site, thereby accomplishing neural stimulation through electrodes with great operational freedom.

Additionally, with the first control module and the second control module respectively controlling the current intensities and frequencies of the first current and the second current, the target site on which the neuron to receive stimulation is situated can be accurately located, thereby providing neural stimulation with high spatial resolution and high precision.

### BRIEF DESCRIPTION OF THE DRAWINGS

FIG. 1 is an applied view of a microelectrode-based neural stimulation system according to a first embodiment of the present invention, showing stimulating electrodes attached to a brain.

FIG. 2A-2B is a schematic drawing illustrating an exemplary principle of stimulation according to the first embodiment of the present invention.

FIG. 3 is a schematic drawing illustrating an exemplary arrangement of the stimulating electrode according to the first embodiment of the present invention.

FIG. 4 is a circuit diagram of the microelectrode-based neural stimulation system according to the first embodiment of the present invention.

FIG. 5 is a schematic drawing illustrating the simulation of neural stimulation according to the first embodiment of the present invention, showing that the stimulating electrodes activate a neuron on a target site.

FIG. 6 is another schematic drawing illustrating simulation of neural stimulation according to the first embodiment of the present invention, showing that the location of the target site can be configured by means of the setting of currents.

FIG. 7 is a schematic drawing illustrating an exemplary arrangement of the stimulating electrode according to a second embodiment of the present invention.

FIG. 8 is a schematic drawing illustrating an exemplary principle of stimulation according to the third embodiment of the present invention.

### DETAILED DESCRIPTION OF THE INVENTION

The following preferred embodiments, when read with the accompanying drawings, are made to clearly exhibit the above-mentioned and other technical contents, features, and effects of the present invention. Through the exposition by means of the specific embodiments, people would further understand the technical means and effects the present invention adopts to achieve the above-indicated objectives. However, the accompanying drawings are intended for reference and illustration, but not to limit the present invention, and are not made to scale.

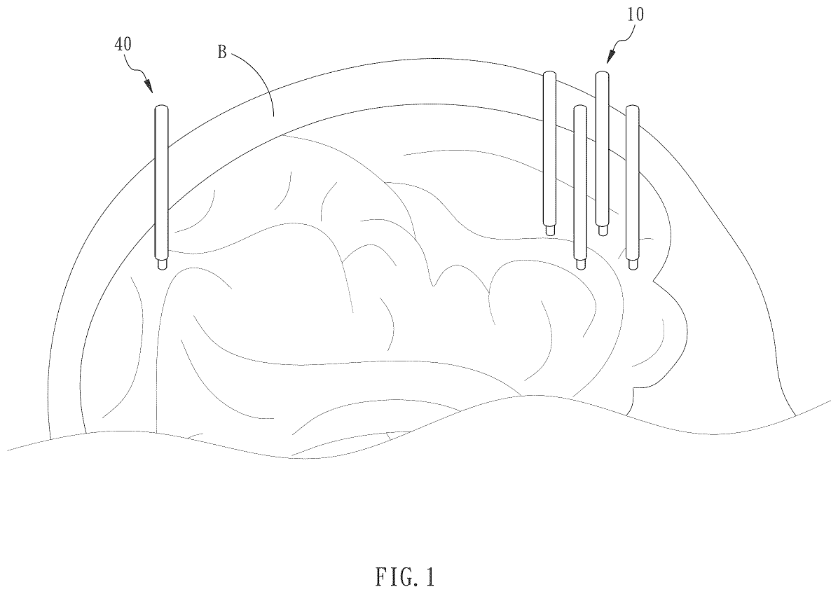

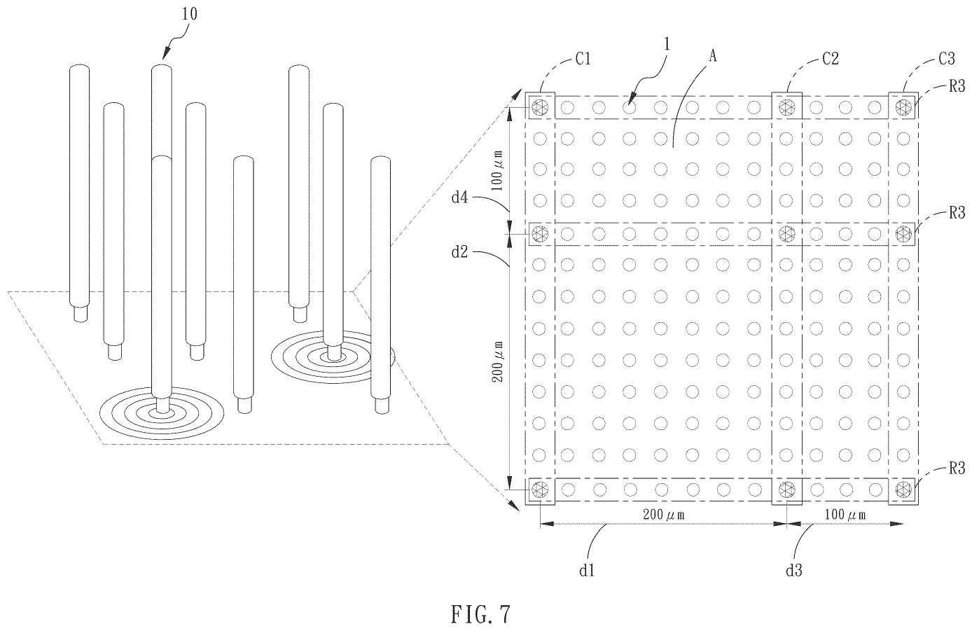

Referring to FIG. 1 through FIG. 8, the present invention provides a high-resolution microelectrode-based neural stimulation system 100, for providing electrical stimulation to a brain tissue B. FIG. 1 through FIG. 6 depict a first embodiment of the present invention and FIG. 7 illustrates a second embodiment of the present invention. In the present invention, the brain tissue B may be located inside a cranium of an animal or may be an acute brain slice obtained from a cranium of an animal.

In the first embodiment, the present invention provides a microelectrode-based neural stimulation system 100, which comprises: four stimulating electrodes 10, a first control module 20, a second control module 30, and a ground electrode 40. Therein, the stimulating electrodes 10 and the ground electrode 40 are fixed to and in contact with the brain tissue B. The first control module 20 and the second control module 30 are coupled to the stimulating electrode 10.

Each of the stimulating electrodes 10 has a columnar shape in the present embodiment and a diameter smaller than 100 μm. In a preferred embodiment, the diameter ranges between 5 and 200 μm. A smaller diameter will be easier to maintain the integrity of the brain tissue B. It should be noted that the stimulating area A can be formed between any two stimulating electrodes 10 if there are more than two stimulating electrodes 10. In some cases, those stimulating electrodes 10 can be arranged in a circular, semicircular, straight or almost straight manner. Moreover, these electrodes 10 do not necessarily need to be arranged in the same plane. Depending on the pattern of brain tissue B, they can be arranged on a curved surface or, if brain tissue B has a three-dimensional structure, those stimulating electrodes 10 can be set at different positions on the same probe to stimulate different depths of brain tissue B, creating a three-dimensional stimulating area A.

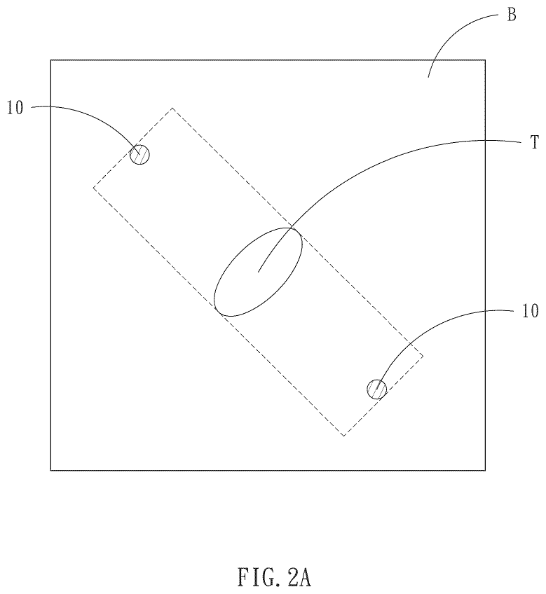

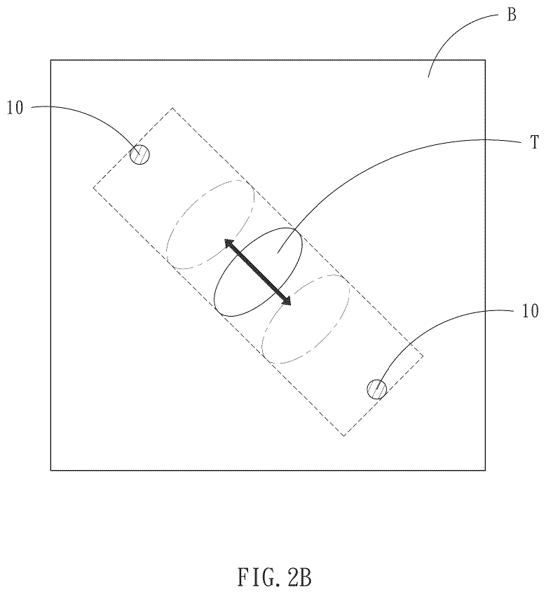

As shown in FIG. 2A, the target position T can be stimulated by controlling the frequencies and current intensities of the two stimulating electrodes 10. For example, when the current intensity of the two stimulating electrodes 10 is the same, the target position T will be located in the middle of the two stimulating electrodes 10. Please refer to FIG. 2B, when the current intensity of the two stimulating electrodes 10 is different, the target position T will move to the side of the stimulating electrode 10 with the lower current intensity. In this way, the user is able to select the desired stimulating target position T by adjusting the current intensity of the stimulating electrode 10.

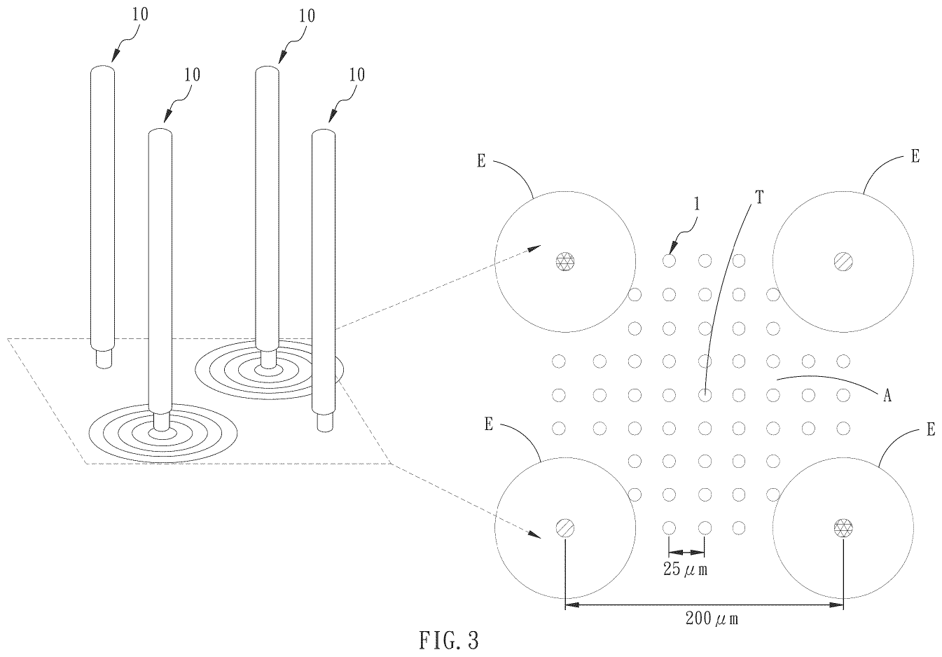

As shown in FIG. 3, this embodiment is illustrated by taking four stimulating electrodes 10 as an example. An overall periphery of all of the four stimulating electrodes 10 as a whole defines a stimulation area A on the brain tissue B. The stimulation area A includes a target site T and an excluded area E. The target site T is a region in which at least two of the stimulating electrodes 10 have resonance with each other. In the drawing, circles in the stimulation area each represent a target site T. In practical use, the distances between and the number of the target sites T may be set and adjusted without limitation. The stimulating electrodes 10 are configured to apply electrical signals, such as currents or voltages, to the brain tissue B for stimulation. However, the intensity and the frequency of the electrical signal tends to be too high around the stimulating electrodes 10 to prevent proper stimulation on the brain tissue B and desired high-frequency oscillation around the stimulating electrodes 10, the areas around the stimulating electrode 10 are each defined as an excluded area E in the present invention. In other words, neural stimulation is impossible for the brain tissue B located in any excluded area E. In the present embodiment, an excluded area E is a region covering a radius of 50 μm around a stimulating electrode 10. The covering radius of the excluded area E should depend on the intensity of the current and neuronal type. More specifically, a preferred radius of the excluded area E ranges between one-fourth to one-third of the distance between the stimulating electrodes 10. Two or more stimulating electrodes 10 can affect a neuron 1 on a target site T with temporal interference (TI) by generating high-frequency wave currents of different intensities. In the present embodiment, the spatial resolution of the target site T is 25 μm. Stated differently, in the stimulation area A, at least one site can be selected for neural stimulation every 25 μm.

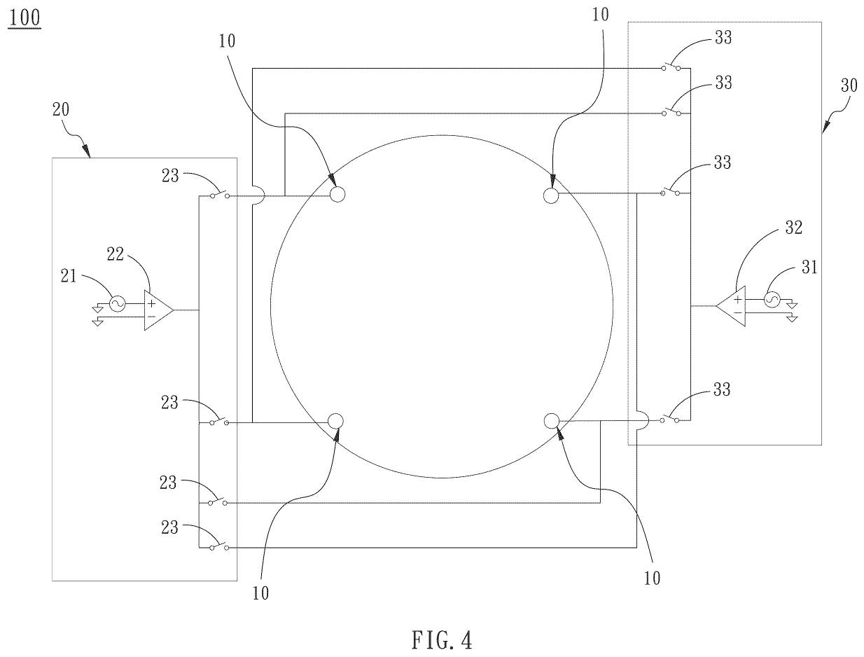

Also referring to FIG. 4, in the present embodiment, the first control module 20 has a first signal source 21, a first signal amplifier 22, and four first switches 23. The first signal source 21 provides a first current having a first frequency through the first signal amplifier 22. Each of the first switches 23 corresponds to a respective stimulating electrode 10 and is electrically connected to the first signal source 21, so as to independently control whether a stimulating electrode 10 is in electric conduction with the first signal source 21.

The second control module 30 has a second signal source 31, a second signal amplifier 32, and four-second switches 33. The second signal source 31 provides a second current having a second frequency through the second signal amplifier 32. Each of the second switches 33 corresponds to a respective stimulating electrode 10 and is electrically connected to second signal source 31, so as to independently control whether a stimulating electrode 10 is in electric conduction with the second signal source 31. Therein, every stimulating electrode 10 is connected to both a first switch 23 and a second switch 33, wherein the first switch 23 and the second switch 33 connected to the same stimulating electrode 10 will not be on at the same time so as to prevent abnormal output of the stimulating electrode 10.

In the present embodiment, the first signal source 21 and the second signal source 31 are each an Analog Discovery 2™ arbitrary waveform generator (Diligent Corporation), and the first signal amplifier 22 and the second signal amplifier 32 are each a power amplifier from BioPro Scientific Corporation.

To provide stimulation to a neuron 1, the first control module 20 controls one of the first switches 23 to establish electric conduction between the corresponding stimulating electrode 10 and the first signal source 21, so as to transmit the first current to the brain tissue B. The second control module 30 controls one of the second switches 33 to establish electric conduction between another stimulating electrode 10 and the second signal source 31, so as to transmit the second current to the brain tissue B.

By controlling the intensities and the frequencies of the first current and the second current, the first control module 20 and the second control module 30 jointly form an electrical stimulation signal on the target site T in stimulation area A so as to provide electrical stimulation to the brain tissue B. In the present embodiment, the electrical stimulation signal may have sine waves or a PWM (Pulse-Width Modulation) waveform, with the sine wave more preferable.

It is to be noted that, in the present embodiment, for preventing mutual interference among the stimulating electrodes 10, only two stimulating electrodes 10 are operated to output electrical stimulation signals to stimulate the neuron 1 at the same time. In other words, the first control module 20 turns on only one of the first switches 23 at one time, and the second control module 30 turns on only one of the second switches 33 at one time, and the stimulating electrode 10 corresponding to the first switch 23 is different from the stimulating electrode 10 corresponding to the second switch 33. Of course, it is feasible to activate plural stimulating electrodes 10 at the same time despite possible temporal interference among the current stimulation signals that may bring about additional complexity.

To be more specific, the second frequency is different from the first frequency. Therein, each of the first frequency and the second frequency is in the range between 800 and 10000 Hz, preferably between 2000 and 4000 Hz. Additionally, the frequency difference between the first frequency and the second frequency ranges between 1 and 500 Hz. An excessively large frequency difference between the first frequency and the second frequency can prevent stimulation from acting on the neurons 1. In the present embodiment, the first frequency is 2000 Hz, and the second frequency is 2040 Hz. In other words, a base frequency of the first frequency and the second frequency is 2000 Hz, and a modulation frequency is the differences between the two frequencies, which is 40 Hz.

Also, the values of the first current and the second current have to be within a defined range because a current of an excessively large or small value may fail to interact with the neurons 1 and prevent the neurons 1 from being stimulated and generating an action potential. Moreover, an excessively high current can lead to water electrolysis, which may break or damage the brain tissue B. In the present embodiment, each of the first current and the second current ranges between 100 and 500 μA.

Furthermore, the first current and the second current have to gradually boost the currents acting on the neuron 1 for stimulation through a period of 50 ms. Such a current ramping process effectively inhibits the onset action potential. In the present embodiment, the ramping rate of the first current and the second current is up to 5 μA/ms.

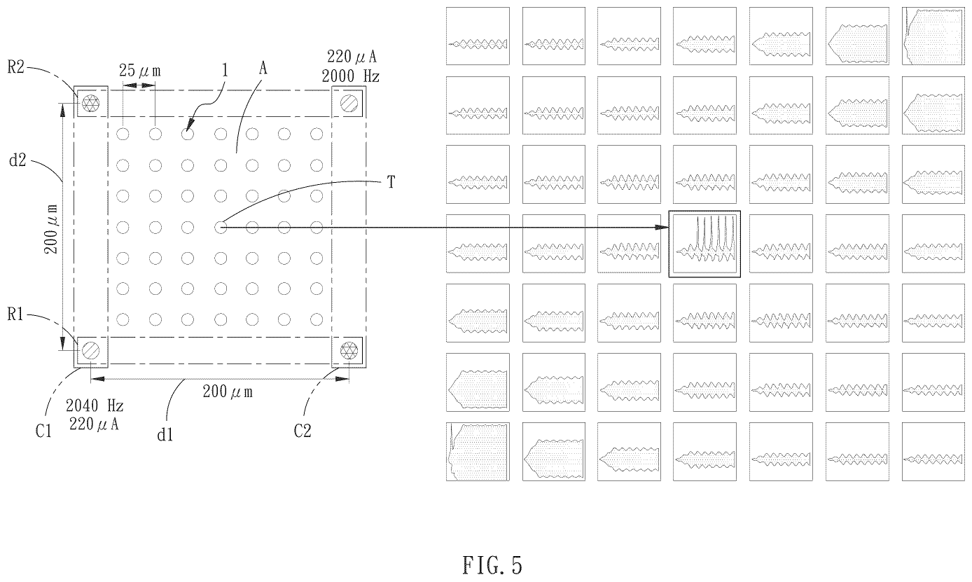

As shown in FIG. 5, in the present embodiment, the four stimulating electrodes 10 are arranged into a two-by-two two-dimensional array, which is composed of, from left to right, a first column C1 and a second column C2, and from the bottom to the top, a first row R1 and a second row R2. Any two adjacent stimulating electrodes 10 are separated. Specifically, the first column C1 and the second column C2 are separated by a first distance d1, and the first row R1 and the second column R2 are separated by a second distance d2. Each of the first distance d1 and the second distance d2 ranges between 50 and 1000 μm. In the present embodiment, each of the first distance d1 and the second distance d2 is 200 μm.

The ground electrode 40 is located far away from the stimulating electrodes 10 (as shown in FIG. 1), so as to prevent electric leakage. In the present embodiment, the ground electrode 40 is separated from any of the stimulating electrodes 10 by a distance that is eight to fifteen times as large as the first distance d1.

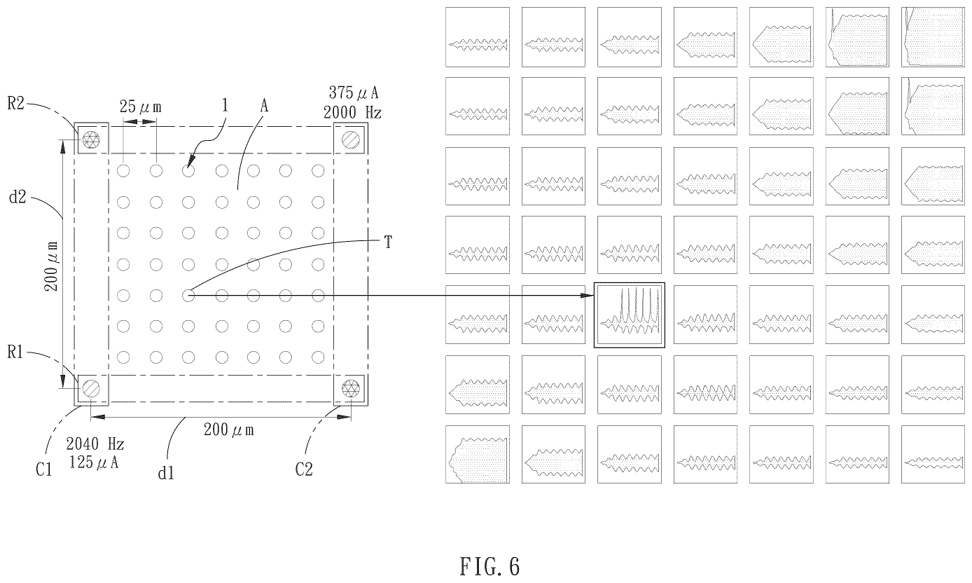

Referring to FIG. 5 and FIG. 6, to evaluate the stimulation effect of the disclosed neural stimulation system 100, simulation waveforms were generated using PYTHON in an electric field in an actual brain, with the simulation environment simplified using LFPY and NEURON.

For the simulation, the first frequency of the first signal source 21 was set as 2040 Hz, and the second frequency of the second signal source 31 was set as 2000 Hz. The stimulating electrodes 10 so obtained were arranged into a two-by-two two-dimensional array to define the stimulation area A, with the first distance d1 and the second distance d2 each set as 200 μm.

While only simulation results in the central part of the stimulation area A are shown, neural stimulation can actually be performed on any target site T located on any straight line linking the stimulating electrodes 10. In the present embodiment, the area for simulation was divided into 49 (7 by 7) equal-size neurons arranged in grid. After the variation and inorganization of neurons 1 were removed, the 49 neurons 1 (ball-and-stick neurons) were distributed across the grid, so that any two adjacent neurons 1 were separated by 25 μm.

As shown in FIG. 5, the electrodes 10 for stimulation were the one at the position (C1, R1), and the one at the position (C2, R2), while the center of the stimulation area A was set as a target site T.

By conducting the stimulating electrode 10 at the position (C1, R1) with the first signal source 21, and conducting the stimulating electrode 10 at the position (C2, R2) with the second signal source 31, while having the first current and the second current set as 220 μA, the stimulating electrode 10 at the position (C1, R1) outputted sinusoidal waves of 2040 Hz, 220 μA, and the stimulating electrode 10 at the position (C2, R2) outputted sinusoidal waves of 2000 Hz, 220 μA.

As indicated by the membrane potentials of the neurons 1 in the 49 neurons arranged in grid, the neuron 1 on the target site T was stimulated and fluctuated obviously (with pulses). The neurons 1 near the stimulating electrode 10 at the position (C1, R2) and the stimulating electrode 10 at the position (C2, R1) that did not output any current had their membrane potentials remaining low and relatively stable. The neurons 1 near the stimulating electrode 10 at the position (C1, R1) and the stimulating electrode 10 at the position (C2, R2) that were conducted stimulation showed membrane potentials that were relatively high but relatively stable. As demonstrated, the stimulating electrodes 10 can perform stimulation accurately on a neuron 1 on a target site T, without affecting non-target neurons 1.

A further simulation was conducted in an electric field in an actual brain for evaluating control of target sites T. As shown in FIG. 6, the stimulating electrode 10 at the position (C1, R1), and the stimulating electrode 10 at the position (C2, R2) were again selected for stimulation in this simulation.

When the first current was changed to 125 μA, and the second current was changed to 375 μA, the stimulating electrode 10 at the position (C1, R1) outputted sinusoidal waves of 2040 Hz, 125 μA, and the stimulating electrode 10 at the position (C2, R2) outputted sinusoidal waves of 2000 Hz, 375 μA. As indicated by the membrane potentials of the neurons 1 in the 49 neurons arranged in grid, the target site T fluctuating obviously had moved from the center toward the stimulating electrode 10 at the position (C1, R1) that had a relatively small output current. This demonstrated that the stimulating electrode 10 can select the location of the target site T according to the location of the neuron 1 to receive stimulation by controlling the current intensities of the first current and the second current. It is also to be noted that since the attenuation of the current intensity with respect to the distance is not linear, the intensity ratio between the first current and the second current is not necessarily equal to the distance ratio between the distances from the target site T to each of the two stimulating electrodes 10.

In a second embodiment, the present invention provides another microelectrode-based neural stimulation system 100, which is similar to its counterpart as described in the first embodiment except that the number of the stimulating electrodes 10 is nine. Therefore, the first control module 20 has nine first switches 23, and the second control module 30 has nine second switches 33. Therein, every stimulating electrode 10 is connected to a first switch 23 and a second switch 33.

Referring to FIG. 7, in the present embodiment, the nine stimulating electrodes 10 are arranged into a three-by-three two-dimensional array composed of, from left to right, a first column C1, a second column C2, and a third row C3, and, from the bottom to the top, a first row R1, a second row R2, and a third column R3. the first column C1 and the second column C2 are separated by a first distance d1. The first row R1 and the second row R2 are separated by a second distance d2. The second column C2 and the third row C3 are separated by a third distance d3. The second row R2 and the third column R3 are separated by a fourth distance d4. Each of the first distance d1, the second distance d2, the third distance d3, and the fourth distance d4 ranges between 50 and 1000 μm. Therein, the first distance d1 is greater than the third distance d3. The first distance d1 is equal to the second distance d2. The third distance d3 is equal to the fourth distance d4. In the present embodiment, the first distance d1 and the second distance d2 are each 200 μm, while the third distance d3 and the fourth distance d4 are each 100 μm.

It is to be noted that since the target site T is determined by the locations of the stimulating electrodes 10 and the current intensities, and in the present embodiment, there are two stimulating electrodes 10 providing currents at the same time, the target site T is on the straight line linking the two stimulating electrodes 10 providing currents. In the present embodiment, with the increased number of the stimulating electrodes 10, which is now nine, the arbitrariness of selecting the target site in the stimulation area A can be significantly increased. Thus, with the increased number of the stimulating electrodes 10 and the selected distances between the stimulating electrodes 10, the scope of the stimulation area A can be expanded and more combinations of the stimulating electrodes 10 can be provided, thereby significantly increasing possible locations of the target site T in the stimulation area A.

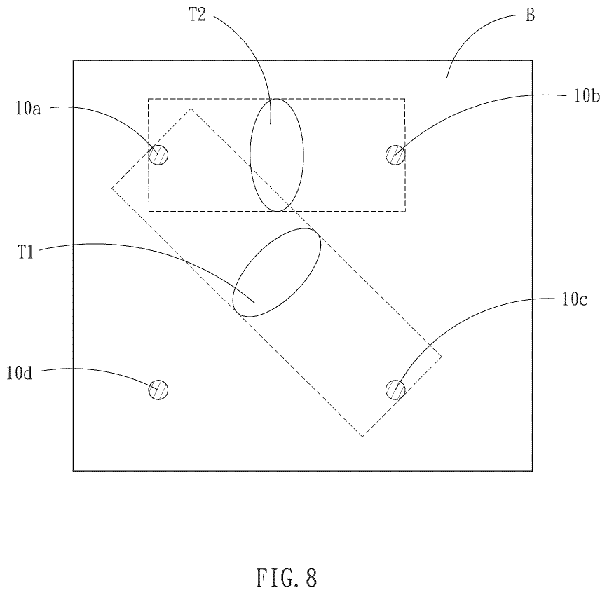

Referring to FIG. 8, these stimulating electrodes 10 can simultaneously stimulate different locations through frequency control. In the present embodiment, stimulating electrode 10a and stimulating electrode 10b can be considered as a first pair of electrodes, with the frequency of stimulating electrode 10a set to 1005 Hz and the frequency of stimulating electrode 10b set to 1040 Hz to stimulate neurons at target position T1. Similarly, stimulating electrode 10a and stimulating electrode 10c can be considered as a second pair of electrodes, with the frequency of stimulating electrode 10a set to 2005 Hz and the frequency of stimulating electrode 10c set to 2040 Hz to stimulate neurons at target position T2. Stimulating electrode 10a can emit current signals with frequencies of 1005 Hz and 2005 Hz during different time periods, corresponding to stimulating electrode 10b and stimulating electrode 10c, respectively, to stimulate neurons at target position T1 and target position T2, respectively. Since the frequency difference between stimulating electrode 10b and stimulating electrode 10c is more than 500 Hz, there is no interference between the first pair of electrodes and the second pair of electrodes. Similarly, stimulating electrode 10b and stimulating electrode 10d can also stimulate neurons and may be combined with other stimulating electrodes 10a and 10c for superimposed stimulation at the same neuron location. To sum up, the present invention at least provides the following beneficial effects:
1. With the designed arrangement of the at least four stimulating electrodes 10, the disclosed microelectrode-based neural stimulation system can use the first control module 20 and the second control module 30 to respectively control any two of the stimulating electrodes 10, so as to form an electrical stimulation signal on a target site T in a stimulation area A and perform stimulation on a neuron on the target site T, thereby accomplishing neural stimulation through electrodes with great operational freedom.
2. With the first control module 20 and the second control module 30, respectively controlling the current intensities of the first current and the second current, the target site on which the neuron 1 to receive stimulation is situated can be accurately located, thereby providing neural stimulation with high spatial resolution and high precision.
3. By setting the ramping rate of the first current and the second current up to 5 μA/ms, the onset action potential can be effectively prevented if not eliminated.
4. By increasing the number of the stimulating electrodes 10 to nine and arranging them as described in Embodiment 2, the arbitrariness of selecting the target site in the stimulation area A can be significantly increased.
5. By separating the ground electrode 40 away from the stimulating electrodes 10, the distance between the ground electrode 40 and any of the stimulating electrodes 10 is eight to fifteen times as large as the first distance d1, so as to prevent electric leakage.
6. Since the arrangement of first distance d1 is not equal to the third distance d3, and the second distance d2 is not equal to the fourth distance d4, the flexibility of target site selection can be increased.

The present invention has been described with reference to the preferred embodiments and it is understood that the embodiments are not intended to limit the scope of the present invention. Moreover, as the contents disclosed herein should be readily understood and can be implemented by a person skilled in the art, all equivalent changes or modifications which do not depart from the concept of the present invention should be encompassed by the appended claims.

What is claimed is:

1. A high-resolution microelectrode-based neural stimulation system for providing electrical stimulation to a brain tissue, the microelectrode-based neural stimulation system comprising:
at least two stimulating electrodes, being fixed to and contacting the brain tissue so that between two stimulating electrodes defines a stimulation area on the brain tissue;
a first control module, having a first signal source and at least two first switches, the first signal source providing a first current having a first frequency, each of the first switches corresponding to a respective one of the stimulating electrodes and being electrically connected to the first signal source, so as to respectively control each of the stimulating electrodes to form electric conduction with the first signal source; and
a second control module, having a second signal source and at least two second switches, the second signal source providing a second current having a second frequency that is different from the first frequency, each of the second switches corresponding to a respective one of the stimulating electrodes and being electrically connected to the second signal source, so as to respectively control each of the stimulating electrodes to form electric conduction with the second signal source;
whereby the first control module controls one of the first switches to form electric conduction between the corresponding stimulating electrode and the first signal source, so as to transmit the first current to the brain tissue, and the second control module controls one of the second switches to form electric conduction between another of the stimulating electrodes and the second signal source, so as to transmit the second current to the brain tissue, in which the first control module and the second control module jointly form an electrical stimulation signal on a target site in the stimulation area by controlling current intensities of the first current and the second current so as to provide the electrical stimulation to the brain tissue.

2. The microelectrode-based neural stimulation system of claim 1, wherein each of the first frequency and the second frequency is in a frequency range between 800 and 10000 Hz.

3. The microelectrode-based neural stimulation system of claim 2, wherein each of the first frequency and the second frequency is in the frequency range between 2000 and 4000Hz.

4. The microelectrode-based neural stimulation system of claim 1, wherein a frequency difference between the first frequency and the second frequency ranges between 1 and 500 Hz.

5. The microelectrode-based neural stimulation system of claim 1, wherein each of the first current and the second current is in a current range between 100 and 500 μA.

6. The microelectrode-based neural stimulation system of claim 1, wherein each of the first current and the second current has a current ramping rate up to 5 μA/ms.

7. The microelectrode-based neural stimulation system of claim 1, wherein those stimulating electrodes are four stimulating electrodes arranged into a two-by-two two-dimensional array that is composed of a first column, a second column, a first row, and a the second row that the first column and the second column are separated by a first distance while the first row and the second row are separated by a second distance, in which each of the first distance and the second distance ranges between 50 and 1000 μm.

8. The microelectrode-based neural stimulation system of claim 7, wherein each of the first distance and the second distance is 200 μm.

9. The microelectrode-based neural stimulation system of claim 7, further comprising a ground electrode, which is separated from any of the stimulating electrodes by a distance that is eight to fifteen times as large as the first distance.

10. The microelectrode-based neural stimulation system of claim 1, wherein those stimulating electrodes are nine stimulating electrodes arranged into a three-by-three two-dimensional array that is composed of a first column, a second column, a third column, a first row, the second row, and a third row that the first column and the second column are separated by a first distance, the first row and the second row are separated by a second distance, the second column and the third row are separated by a third distance, and the second row and the third column are separated by a fourth distance.

11. The microelectrode-based neural stimulation system of claim 10, wherein each of the first distance, the second distance, the third distance, and the fourth distance ranges between 50 and 1000 μm.

12. The microelectrode-based neural stimulation system of claim 11, wherein the first distance is greater than the third distance, the first distance is equal to the second distance, and the third distance is equal to the fourth distance.

13. The microelectrode-based neural stimulation system of claim 12, wherein each of the first distance and the second distance is 200 μm, and each of the third distance and the fourth distance is 100 μm.

14. The microelectrode-based neural stimulation system of claim 1, wherein each of the stimulating electrodes has a columnar shape, and a diameter ranging between 5 and 200 μm.

15. The microelectrode-based neural stimulation system of claim 1, wherein an excluded area is defined in the stimulation area and is located in a radius of 50 μm around each of the stimulating electrodes.

16. The microelectrode-based neural stimulation system of claim 1, wherein the brain tissue is located inside a cranium of an animal or is an acute brain slice obtained from a cranium of an animal.
